(19) United States
(12) Patent Application Publication
(10) Pub. No.: US 2024/0060144 A1
(43) Pub. Date: Feb. 22, 2024

(54) Composition and method for predicting preterm birth using microbiome analysis of vaginal fluid of pregnant women

(71) Applicant: EWHA University Industry Collaboration Foundation, Seoul (KR)

(72) Inventors: Young Ju KIM, Seoul (KR); Sun Wha PARK, Seoul (KR); Young Ah YOU, Seoul (KR); Soo Min KIM, Incheon-si (KR); Ga In LEE, Seoul (KR); Suenie Park, Seoul (KR)

(21) Appl. No.: 18/093,092

(22) Filed: Jan. 4, 2023

(30) Foreign Application Priority Data
Aug. 22, 2022 (KR) 10-2022-0105109

(51) Int. Cl. C12Q 1/689

(52) U.S. Cl. CPC C12Q 1/689; C12Q 1/6869

(57) ABSTRACT

One aspect relates to a composition for predicting preterm birth of the pregnant woman comprising an agent for detecting microorganisms of at least one genus selected from the group consisting of the genus Ureaplasma and the genus Prevotella and microorganisms of the genus Lactobacillus in a vaginal fluid sample of the pregnant woman and a method for predicting preterm birth using the same. According to one aspect, if the relative abundance ratio of the detected microorganisms of at least one genus selected from the group consisting of the genus Ureaplasma and the genus Prevotella is higher than that of normal pregnant woman, and the relative abundance ratio of the detected microorganisms of the genus Lactobacillus is lower than that of normal pregnant woman, it is predicted that preterm birth will occur. Therefore, the composition can be usefully used to predict preterm birth in pregnant women.

### TECHNICAL FIELD

The present invention relates to a composition for predicting preterm birth using microbiome analysis of vaginal fluid of pregnant women and a method for predicting preterm birth.

### REFERENCE TO AN ELECTRONIC SEQUENCE LISTING

The content of the electronic sequence listing (OP23-0002HSUS_SEQLIST.XML; Size: 4,114 bytes; and Date of Creation: Mar. 14, 2023), filed via Patent Center on Mar. 14, 2023, is herein incorporated by reference in its entirety.

### BACKGROUND ART

Preterm birth (PTB) means delivery within 37 weeks of pregnancy. Newborns born prematurely account for about 60% of all infant deaths and even in the case of surviving infants, neonatal intensive care is required due to nervous system development disorders, respiratory or cardiovascular complications, hearing or vision loss, cerebral palsy, and postnatal growth retardation, and furthermore, the morbidity rate of serious long-term or short-term diseases is high.

It is known that spontaneous preterm birth due to preterm labor and preterm premature rupture of membrane accounts for about 70% of all preterm births. The exact causes of preterm labor are not known, but among them ⅓ is known to be related to intrauterine infection, and preterm labor and preterm birth rates are expected to increase further due to the increase in older pregnancies and the increase in multiple pregnancies due to the development of assisted reproductive technologies.

Until now, for the treatment of preterm labor, uterine contraction inhibitors, antibiotic treatment, and steroid and progesterone administration have been used to suppress labor and delay delivery but it is known that the therapeutic effect of antibiotics and other treatments for intrauterine infection and inflammation is very limited and it is difficult to prevent preterm birth in cases where preterm labor has already begun or premature rupture of membranes has occurred.

Therefore, rather than performing treatment after symptoms of premature membrane rupture or preterm labor appear, it is necessary to develop effective treatment methods of preterm birth and prevent preterm birth by applying the system or based on the analysis results of the causes of preterm birth by applying techniques that can predict the risk of preterm birth in advance, selecting a group of pregnant women at risk and managing them more systematically and intensively. In addition, the earlier the timing of preterm birth, the higher the possibility of leaving sequelae in the newborn and the severity of the sequelae. Therefore, if preterm birth can be predicted including measures to delay preterm birth, the incidence of premature babies and consequently disabled children are expected to be significantly reduced.

Many studies have been conducted to discover biochemical markers to predict sudden preterm birth or membrane rupture, substances such as plasma estradiol-17 beta, progesterone, and C-reactive protein have been nominated as candidates, but the accuracy of those markers were not satisfactory, and its efficacy was not clear. Therefore, it is necessary to develop markers which is easier to detect with high sensitivity and specificity.

Accordingly, a composition for predicting preterm birth and a method for predicting preterm birth were developed by identifying microbes related to preterm birth or late birth through microbiome analysis of pregnant woman's vaginal fluid, and by identifying microorganisms that increase during full-term birth, it was developed that these microorganisms may have an effect of preventing preterm birth.

### PRIOR ART DOCUMENT

### Patent Document

Korea Patent Registration No. 10-2329189

### DISCLOSURE

### Technical Problem

One aspect is to provide a composition for prediction including an agent for detecting microorganisms of at least one genus selected from the group consisting of the genus Ureaplasma and the genus Prevotella and microorganisms of the genus Lactobacillus in a vaginal fluid sample of the pregnant woman.

Another aspect is to provide a kit for prediction including an agent for detecting microorganisms of at least one genus selected from the group consisting of the genus Ureaplasma and the genus Prevotella and microorganisms of the genus Lactobacillus in a vaginal fluid sample of the pregnant woman.

Another aspect is to provide an information providing method for predicting preterm birth of pregnant women comprising steps to detect microorganisms which consisted of at least one organism of genus selected from either group of Ureaplasma and Prevotella and Lactobacillus in vaginal fluid samples of pregnant women.

Another aspect is to provide a method for diagnosing preterm birth in pregnant women comprising the steps to detect microorganisms which consisted of at least one organism of genus or more selected from either group of Ureaplasma and Prevotella and Lactobacillus in vaginal fluid samples of pregnant women.

Another aspect is to provide a use of a preparation for detecting microorganisms which consisted of at least one organism of genus or more selected from either group of Ureaplasma and Prevotella and Lactobacillus in vaginal fluid samples of pregnant women to produce a drug for diagnosing preterm birth in pregnant women.

### Technical Solution

One aspect is to provide a composition for predicting preterm birth of a pregnant women which comprised of at least one organism of genus selected from group of Ureaplasma, Prevotella and Lactobacillus in vaginal fluid samples of pregnant women.

The above term “pregnant woman” means a woman who is pregnant with a baby, and the above term “Preterm birth” means delivery within 37 weeks of gestation. For newborns born prematurely, the risk of death is high enough to account for about 60% of all infant deaths. It is known that natural preterm birth due to preterm labor and premature rupture of membranes accounts for about 70% or more of all preterm birth.

“Term birth” or “full term birth” refers to delivery between 37 and 42 weeks of gestation and if delivery occurs after 42 weeks of gestation it means “delayed pregnancy” or “delivery after full term”. In this case, the newborn is referred to as a “postmature baby”.

The above term “vaginal fluid sample” refers to a cell sample or a tissue sample taken from the vagina.

and the vaginal fluid is specifically cervicovaginal fluid.

The microorganism of the genus Ureaplasma means a microorganism composed of species belonging to the genus Ureaplasma taxonomically. The microorganisms of the genus Ureaplasma may include microorganisms having 70% or more, 75% or more, 80% or more, 85% or more, 90% or more, 95% or more, 96% or more, 97% or more, 98% or more, or 99% or more of nucleic acid sequence homology of previously reported microorganisms of the genus Ureaplasma.

The above term “Homology” refers to a similarity with a wild-type nucleic acid sequence. The term “homology” is intended to indicate the degree of similarity with a wild-type nucleic acid sequence, and comparison of such homology can be performed using a comparison program widely known in the art, and homology between two or more sequences can be calculated as a percentage (%).

The microorganism of the genus Prevotella means a microorganism composed of species belonging to the genus Prevotella taxonomically. The microorganisms of the genus Prevotella may include microorganisms having 70% or more, 75% or more, 80% or more, 85% or more, 90% or more, 95% or more, 96% or more, 97% or more, 98% or more, or 99% or more of nucleic acid sequence homology of previously reported microorganisms of the genus Prevotella.

The microorganism of the genus Lactobacillus means a microorganism composed of species belonging to the genus Lactobacillus taxonomically. The microorganisms of the genus Lactobacillus may include microorganisms having 70% or more, 75% or more, 80% or more, 85% or more, 90% or more, 95% or more, 96% or more, 97% or more, 98% or more, or 99% or more of nucleic acid sequence homology of previously reported microorganisms of the genus Lactobacillus.

The term “prediction of preterm birth of a pregnant woman” means determining whether the pregnant woman is likely to give birth prematurely or show signs of preterm birth.

In one aspect, the agent or preparation may further comprise an agent to detect microorganisms of at least one genus selected from the group consisting of the genus Staphylococcus and the genus Escherichia, or the composition may further comprise an agent to detect microorganisms of at least one genus selected from the group consisting of the genus Staphylococcus and the genus Escherichia.

The microorganism of the genus Staphylococcus means a microorganism composed of species belonging to the genus Staphylococcus taxonomically. The microorganisms of the genus Staphylococcus may include microorganisms having 70% or more, 75% or more, 80% or more, 85% or more, 90% or more, 95% or more, 96% or more, 97% or more, 98% or more, or 99% or more of nucleic acid sequence homology of previously reported microorganisms of the genus Staphylococcus.

The microorganism of the genus Escherichia means a microorganism composed of species belonging to the genus Escherichia taxonomically. The microorganisms of the genus Escherichia may include microorganisms having 70% or more, 75% or more, 80% or more, 85% or more, 90% or more, 95% or more, 96% or more, 97% or more, 98% or more, or 99% or more of nucleic acid sequence homology of previously reported microorganisms of the genus Escherichia.

In one aspect, the agent can detect the relative abundance ratio of microorganisms of at least one genus selected from the group comprising of the genus Ureaplasma and Prevotella, and microorganisms of at least one genus selected from the group comprising of the genus Staphylococcus and the genus Escherichia, and microorganisms of the genus Lactobacillus.

The term “relative abundance” refers to the ratio of a specific species within the microbiome present in a sample.

In one aspect, the agent can detect the relative abundance ratio of microorganisms of at least one genus selected from the group comprising of the genus Ureaplasma and Prevotella, and microorganisms of at least one genus selected from the group comprising of the genus Staphylococcus and the genus Escherichia, and microorganisms of the genus Lactobacillus and preterm birth can be predicted by comparing the detected relative abundance with that of normal pregnant women.

Specifically, if the relative abundance ratio of microorganisms of at least one genus selected from the group comprising of the genus Ureaplasma and the genus Prevotella is lower than that of normal pregnant women, and the relative abundance ratio of microorganisms of at least one genus selected from the group comprising of the genus Staphylococcus and Escherichia in the vaginal fluid sample of pregnant women is lower than that of normal pregnant women and the relative abundance ratio of microorganisms of the genus Lactobacillus in the vaginal fluid sample of pregnant women is higher than that of normal pregnant women, it can be predicted that full-term labor will occur;

if the relative abundance ratio of microorganisms of at least one genus selected from the group comprising of the genus Ureaplasma and the genus Prevotella in the vaginal fluid sample of pregnant women is higher than that of normal pregnant women, and the relative abundance ratio of microorganisms of at least one genus selected from the group comprising of the genus Staphylococcus and Escherichia is higher than that of normal pregnant women and the relative abundance ratio of microorganisms of the genus Lactobacillus is lower than that of normal pregnant women, it can be predicted that preterm birth will occur. Therefore, the composition can be usefully used for rapid and accurate prediction of preterm birth in pregnant women.

The “normal pregnant woman” means a pregnant woman whose full term is predicted. Specifically, when the relative abundance ratio of microorganisms of the genus Ureaplasma, microorganisms of the genus Prevotella, microorganisms of the genus Staphylococcus, and microorganisms of the genus Escherichia in vaginal fluid samples of pregnant women was less 0.5%, respectively and the relative abundance ratio of microorganisms of the genus Lactobacillus is 80% or more, the pregnant woman can be predicted as a normal pregnant woman.

For example, compared to the normal pregnant woman, when the relative abundance ratio of microorganisms of one or more genera selected from the group consisting of the genus Ureaplasma and the genus Prevotella in the vaginal fluid sample of the pregnant woman and the relative abundance ratio of microorganisms of one or more genera selected from the group consisting of the genus Staphylococcus and the genus Escherichia in vaginal fluid samples of pregnant women is higher than normal pregnant women, and the relative abundance ratio of microorganisms of the genus Lactobacillus is lower than normal pregnant women, that is, if the relative abundances ratio of microorganisms of the genus Ureaplasma, microorganisms of the genus Prevotella, and microorganisms of the genus Staphylococcus in the vaginal fluid samples of pregnant women are each more than 2%, the relative abundance ratio of microorganisms of the genus Escherichia is more than 1%, and the relative abundance ratio of microorganisms of the genus Lactobacillus is less than 70%, the pregnant woman may be expected to give birth prematurely.

In one aspect, the “agent to detect” means a substance that can be used to detect the presence of microorganisms of one or more genera is selected from the group consisting of Ureaplasma genus, Prevotella genus, Staphylococcus genus, and Escherichia and genus of the genus Lactobacillus in the sample For example, it means a substance capable of specifically detecting organic biomolecules such as proteins, nucleic acids, lipids, glycolipids, glycoproteins or sugars (monosaccharides, disaccharides, oligosaccharides, etc.) that are specifically present in the microorganisms or produced by the microorganisms, and, specifically, the agent may be one or more selected from the group consisting of primers, probes, antisense oligonucleotides, aptamers, and antibodies specific to the microorganisms.

The term “primer” refers to a nucleic acid sequence of 5 to 50 nt. with a short free 3′ hydroxyl group, capable of forming a base pair with a complementary template, and serving as a starting point for template strand copying. The sequence of the primer does not necessarily have to be exactly the same as the sequence of the template but is sufficiently complementary to allow hybridization with the template. Primers can initiate DNA synthesis in the presence of reagents for polymerization (i.e., DNA polymerase or reverse transcriptase and four different nucleoside triphosphates) in an appropriate buffer solution and temperature. PCR conditions and lengths of sense and antisense primers can be modified based on those known in the art.

The term “probe” refers to a substance capable of specifically binding to a target substance to be detected in a sample, and means a substance capable of specifically confirming the presence of a target substance in a sample through the binding. The type of probe is a material commonly used in the art, and may be PNA (peptide nucleic acid), LNA (locked nucleic acid), peptide, polypeptide, protein, RNA, or DNA. Specifically, DNA may include cDNA, genomic DNA, and oligonucleotides, RNA may include genomic RNA, mRNA, and oligonucleotides, and proteins may include antibodies, antigens, enzymes, peptides, and the like.

The term “antibody” refers to a specific protein molecule directed against an antigenic site. The antibody refers to a protein molecule that specifically binds to a microorganism according to one aspect, and the antibody can be produced against a protein encoded by the gene of the microorganism by cloning each gene into an expression vector according to a conventional method, and it can be prepared by a conventional method from the obtained protein. The antibody may include a polyclonal antibody, a monoclonal antibody, or an immunoglobulin antibody, and may also include a special antibody such as a humanized antibody. An antibody against a protein encoded by a gene of a microorganism according to one aspect may be prepared by a method known in the art. In addition, the antibody may include a functional fragment of the antibody molecule as well as a complete form having two full-length light chains and two full-length heavy chains. The functional fragment of the antibody molecule refers to a fragment having at least an antigen-binding function, and may be Fab, F(ab′), F(ab′)₂, Fv, or the like.

Specifically, in one aspect, the agent may be specific for one or more base sequences selected from the group consisting of 16s rRNA base sequence, 18s rRNA base sequence, and ITS (Internal transcribed spacer) base sequence of the microorganisms. And, more specifically, it may be specific to the base sequence of 16s rRNA of the microorganisms.

The “16s rRNA” is an rRNA constituting the 30S subunit of the prokaryotic ribosome, and since there is a conserved region common to all species and a hypervariable region that can classify a specific species, microorganisms can be identified through sequencing. Particularly since there is little diversity among homologous species, but there is diversity between different species, prokaryotes can be usefully identified by comparing 16s rRNA sequences. In addition, since 16s rDNA is a gene encoding 16s rRNA, microorganisms can be identified using 16s rDNA

In one aspect, the agent may further confirm the Shannon α-diversity index, or the composition may further include an agent for confirming the Shannon α-diversity index.

According to one aspect, when the agent can further confirm the Shannon α-diversity index, or when the composition further includes an agent for determining the Shannon α-diversity index, after detecting the relative abundance ratio of microorganisms in the genus microorganisms of at least one genus selected from the group consisting of the genus Ureaplasma and the genus Prevotella, and/or microorganisms of at least one genus selected from the group consisting of the genus Staphylococcus and the genus Escherichia, and the genus Lactobacillus, preterm birth can be predicted more accurately by further comparing the identified Shannon alpha diversity index with the Shannon alpha diversity index of normal pregnant women.

Specifically, if the relative abundance ratio of microorganisms of at least one genus selected from the group consisting of the genus Ureaplasma and the genus Prevotella in the vaginal fluid sample of pregnant women is lower than that of normal pregnant women, and/or the relative abundance ratio of microorganisms of at least one genus selected from the group consisting of the genus Staphylococcus and the genus Escherichia is lower than that of normal pregnant women, and the relative abundance ratio of microorganisms of the genus Lactobacillus is higher than that of normal pregnant women, and furthermore, if the Shannon's alpha diversity index of the vaginal fluid sample in pregnant women is equal to or smaller than the Shannon's alpha diversity index of a normal pregnant woman, it is possible to more accurately predict full term;

if the relative abundance ratio of microorganisms of at least one genus selected from the group consisting of the genus Ureaplasma and the genus Prevotella in the vaginal fluid sample of pregnant women is higher than that of normal pregnant women, and/or the relative abundance ratio of microorganisms of at least one genus selected from the group consisting of the genus Staphylococcus and the genus Escherichia is higher than that of normal pregnant women, and the relative abundance ratio of microorganisms of the genus Lactobacillus is lower than that of normal pregnant women, and furthermore, if the Shannon's alpha diversity index of the vaginal fluid sample in pregnant women is larger than the Shannon's alpha diversity index of a normal pregnant woman, it is possible to more accurately predict preterm birth. Therefore, the composition can be usefully used for more rapid and accurate prediction of preterm birth in pregnant women.

The “normal pregnant woman” means a pregnant woman whose full term is predicted. Specifically, the relative abundance ratio of microorganisms of the genus Ureaplasma, microorganisms of the genus Prevotella, or microorganisms of the genus Staphylococcus, and microorganisms of the genus Escherichia in vaginal fluid samples of pregnant women is less than 0.5%, respectively, and the relative abundance of microorganisms of the genus Lactobacillus is 80% or more, and the Shannon alpha diversity index of the vaginal fluid sample of the pregnant woman is 2 or less, the pregnant woman can be more accurately predicted as a normal pregnant woman.

For example, compared to the normal pregnant woman, if the relative abundance ratio of microorganisms of the genus Ureaplasma, microorganisms of the genus Prevotella, and/or microorganisms of the genus Staphylococcus in the vaginal fluid sample of the pregnant woman is 2% or more, respectively and the relative abundance ratio of microorganisms of the genus Escherichia is 1% or more, the relative abundance ratio of microorganisms of the genus Lactobacillus is less than 70%, and further the Shannon alpha diversity index of vaginal fluid samples of pregnant women is larger than the Shannon alpha diversity index of a normal pregnant woman, that is, if the median value based on the interquartile range of the Shannon alpha diversity index of the pregnant woman's vaginal fluid sample is 2.1 or more, the pregnant woman may be a pregnant woman who is expected to give birth prematurely.

According to one embodiment, 102 out of a total of 203 subjects were classified into a PTB (pre-term birth) group, and 101 subjects were classified into a TB (term birth) group, and clinical characteristics were compared. As a result, for the PTB and TB groups, the history of sPTB (spontaneous PTB), WBC (white blood cell) count, CL (cervical length), GAS (gestational age at sampling), GAB (Gestational age at birth), birth weight, skin color, pulse, grimaces, activity, and respiration score (APGAR score), NICU admission rates (neonatal intensive care unit admission), it was confirmed that there were significant differences (see Example 1).

According to another embodiment, as a result of comparing the microbial diversity between the PTB group and the TB group, in the PTB group, Lactobacillus accounted for 69%, Prevotella 2%, and Ureaplasma 2%, but in the TB group, Lactobacillus accounted for 88%. Prevotella accounted for 0.3%, Ureaplasma accounted for 0.2%, Staphylococcus accounted for 0.5%, and Escherichia accounted for less than 0.1%. As a result of Shannon's α-diversity index analysis, the median value (interquartile range) of the PTB group was 2.20 (1.27-2.12), which was significantly higher than the median (interquartile range) of the TB group, 1.99 (1.60-3.03) (see Example 2).

According to another embodiment, as a result of analyzing the related microbiome in each of the PTB group and the TB group, the abundance ratio of microorganisms of the genus Lactobacillus in the TB group was measured to be significantly higher than that of the PTB group, and in the PTB group, it was confirmed that the abundance ratio of microorganisms of the genus Ureaplasma, microorganisms of the genus Prevotella, microorganisms of the genus Staphylococcus, and microorganisms of the genus Escherichia was significantly higher than that of the TB group (see Example 3).

According to another embodiment, as a result of analyzing the correlation between the microbiome in the PTB group and the TB group, Staphylococcus showed a negative correlation of 0.3 with Lactobacillus, in which case it showed an association with TB, and when Ureaplasma showed a negative correlation with Lactobacillus, it showed a high correlation with TB. Similarly, in the case of Prevotella, a negative correlation with Lactobacillus showed a high correlation with TB, and in case than Escherichia showed a negative correlation with Lactobacillus, it was confirmed that the correlation with TB was high (see Example 4).

In another aspect, A kit for prediction of a preterm birth of a pregnant woman comprising an agent for detecting microorganisms of at least one genus selected from the group consisting of the genus Ureaplasma and the genus Prevotella and microorganisms of the genus Lactobacillus in a vaginal fluid sample of the pregnant woman. is provided.

The “vaginal fluid sample”, “Ureaplasma”, “Prevotella”, “Lactobacillus”, “agent”, “to detect”, “preterm birth”, “prediction”, etc. may be within the aforementioned range.

In one aspect, the agent further detects microorganisms of at least one genus selected from the group consisting of Staphylococcus genus and Escherichia genus, or the composition may further comprise an agent for detecting one or more microorganisms selected from the group consisting of the genus Staphylococcus and the genus Escherichia.

According to one aspect, the agent can detect relative abundance ratio of a microorganism of at least one genus selected from the group consisting of the genus Ureaplasma and the genus Prevotella, microorganism of at least one genus selected from the group consisting of the genus Staphylococcus and the genus Escherichia, and microorganisms of the genus Lactobacillus and preterm birth can be predicted by comparing the detected relative abundance ratio with that of normal pregnant women. Therefore, the kit can be usefully utilized for rapid and accurate prediction of preterm birth in pregnant women.

In one aspect, the kit may comprise a test tube or other suitable container, reaction buffer, deoxynucleotides (dNTPs), enzymes such as Taq-polymerase and reverse transcriptase, DNase, RNAse inhibitors, DEPC-water or sterile water, and the like.

In another aspect, a method for providing information for predicting preterm birth in pregnant women comprising detecting microorganisms of at least one genus selected from the group consisting of the genus Ureaplasma and the genus Prevotella and microorganisms of the genus Lactobacillus from a vaginal fluid sample isolated from a pregnant woman is provided.

The “vaginal fluid sample”, “Ureaplasma”, “Prevotella”, “Lactobacillus”, “preterm birth”, “prediction”, etc. may be within the aforementioned range.

In one aspect, the method may comprise extracting nucleic acids from a vaginal fluid sample isolated from a pregnant woman prior to the detecting step.

The extraction may be performed through a known DNA extraction method, and may be performed by chemical or mechanical disruption, dissolution using a surfactant, or a combination thereof. Specifically, the extraction step may be performed through a DNA extraction process according to MobioPowerSoil®, a FASTDNA® SPIN kit or a NucleoSpin® kit, and more specifically, a NucleoSpin® kit.

In one aspect, the method may further comprise amplifying the extracted nucleic acid after the extracting step and before the detecting step.

The amplification step can be specifically carried out through polymerase chain reaction (PCR), reverse transcription-polymerase chain reaction (RT-PCR), multiplex PCR, touchdown PCR, hot start PCR, nested PCR , booster PCR, real-time PCR, differential display PCR (DD-PCR), rapid amplification of cDNA ends (RACE), inverse polymerase chain Reaction, vectorette PCR, thermal asymmetric interlaced PCR (TAIL-PCR), ligase chain reaction, repair chain reaction, transcription-mediated amplification, replication of self-maintained sequences, or selective amplification of target sequences.

According to one aspect, the method can detect the relative abundance ratio of microorganisms of at least one genus selected from the group consisting of the genus Ureaplasma and the genus Prevotella and the microorganisms of the genus Lactobacillus, and preterm birth can be predicted by comparing the detected relative abundance ratio with that of normal pregnant women.

Specifically, the method may further comprise predicting step wherein if the relative abundance ratio of detected microorganisms of one or more genera selected from the group consisting of the genus Ureaplasma and the genus Prevotella is lower than that of normal pregnant women, and the relative abundance ratio of microorganisms of the detected Lactobacillus genus is higher than that of normal pregnant women, it is predicted to be full-term, and

if the relative abundance ratio of detected microorganisms of one or more genera selected from the group consisting of the genus Ureaplasma and the genus Prevotella is higher than that of normal pregnant women, and the relative abundance ratio of microorganisms of the detected Lactobacillus genus is lower than that of normal pregnant women, it is predicted to be preterm birth.

Also, according to one aspect, the method may further comprise steps for detecting microorganisms of one or more selected genera in the group consisting of Staphylococcus genus and Escherichia genus from the sample.

In one aspect, the method may further comprise steps wherein if the relative abundance ratio of detected microorganisms of one or more genera selected from the group consisting of the genus Ureaplasma and the genus Prevotella is lower than that of normal pregnant women,
the relative abundance ratio of detected microorganisms of one or more selected genera in the group consisting of Staphylococcus genus and Escherichia genus is lower than that of normal pregnant women and
the relative abundance ratio of microorganisms of the detected Lactobacillus genus is higher than that of normal pregnant women, it is predicted to be full-term, and
if the relative abundance ratio of detected microorganisms of one or more genera selected from the group consisting of the genus Ureaplasma and the genus Prevotella is higher than that of normal pregnant women,
the relative abundance ratio of detected microorganisms of one or more selected genera in the group consisting of Staphylococcus genus and Escherichia genus is higher than that of normal pregnant women and
the relative abundance ratio of microorganisms of the detected Lactobacillus genus is lower than that of normal pregnant women, it is predicted to be preterm birth. Therefore, the method can be usefully utilized for rapid and accurate prediction of preterm birth in pregnant women.

The “normal pregnant woman” means a pregnant woman whose full term is predicted. Specifically, when the relative abundance ratio of microorganisms of the genus Ureaplasma, microorganisms of the genus Prevotella, microorganisms of the genus Staphylococcus, and microorganisms of the genus Escherichia in vaginal fluid samples of pregnant women was less than 0.5%, respectively and the relative abundance ratio of microorganisms of the genus Lactobacillus is 80% or more, the pregnant woman can be predicted to expect full-term birth.

For example, compared to the normal pregnant woman, when the relative abundance ratio of microorganisms of one or more genera selected from the group consisting of the genus Ureaplasma and the genus Prevotella in the vaginal fluid sample of the pregnant woman and the relative abundance ratio of microorganisms of one or more genera selected from the group consisting of the genus Staphylococcus and the genus Escherichia in vaginal fluid samples of pregnant women is higher than normal pregnant women, and the relative abundance ratio of microorganisms of the genus Lactobacillus is lower than normal pregnant women, that is, if the relative abundances ratio of microorganisms of the genus Ureaplasma, microorganisms of the genus Prevotella, and microorganisms of the genus Staphylococcus in the vaginal fluid samples of pregnant women are more than 2% each, the relative abundance ratio of microorganisms of the genus Escherichia is more than 1%, and the relative abundance ratio of microorganisms of the genus Lactobacillus is less than 70%, the pregnant woman may be expected to give birth prematurely.

In one aspect, the method may further comprise identifying a Shannon α-diversity index from the sample and by comparing the identified Shannon α-diversity index with the Shannon α-diversity index of a normal pregnant woman, preterm birth can be more accurately predicted.

Specifically, if the relative abundance ratio of microorganisms of at least one genus selected from the group consisting of the genus Ureaplasma and the genus Prevotella in the vaginal fluid sample of pregnant women is lower than that of normal pregnant women, and the relative abundance ratio of microorganisms of at least one genus selected from the group consisting of the genus Staphylococcus and the genus Escherichia is lower than that of normal pregnant women, and the relative abundance ratio of microorganisms of the genus Lactobacillus is higher than that of normal pregnant women, and furthermore, if the Shannon's alpha diversity index of the vaginal fluid sample in pregnant women is equal to or smaller than the Shannon's alpha diversity index of a normal pregnant woman, it is predicted to be full-term;

if the relative abundance ratio of microorganisms of at least one genus selected from the group consisting of the genus Ureaplasma and the genus Prevotella in the vaginal fluid sample of pregnant women is higher than that of normal pregnant women, and the relative abundance ratio of microorganisms of at least one genus selected from the group consisting of the genus Staphylococcus and the genus Escherichia is higher than that of normal pregnant women, and the relative abundance ratio of microorganisms of the genus Lactobacillus is lower than that of normal pregnant women, and furthermore, if the Shannon's alpha diversity index of the vaginal fluid sample in pregnant women is larger than the Shannon's alpha diversity index of a normal pregnant woman, it is predicted to be preterm birth.

The “normal pregnant woman” means a pregnant woman whose full term is predicted. Specifically, the relative abundance ratio of microorganisms of the genus Ureaplasma, microorganisms of the genus Prevotella, microorganisms of the genus Staphylococcus, and microorganisms of the genus Escherichia in vaginal fluid samples of pregnant women is less than 0.5%, respectively, and the relative abundance of microorganisms of the genus Lactobacillus is 80% or more, and the Shannon alpha diversity index of the vaginal fluid sample of the pregnant woman is 2 or less, the pregnant woman can be more accurately predicted as a normal pregnant woman.

For example, compared to the normal pregnant woman, if the relative abundance ratio of microorganisms of the genus Ureaplasma, microorganisms of the genus Prevotella, and microorganisms of the genus Staphylococcus in the vaginal fluid sample of the pregnant woman is 2% or more, respectively and the relative abundance ratio of microorganisms of the genus Escherichia is 1% or more, the relative abundance ratio of microorganisms of the genus Lactobacillus is less than 70%, and further the Shannon alpha diversity index of vaginal fluid samples of pregnant women is larger than the Shannon alpha diversity index of a normal pregnant woman, that is, if the median value based on the interquartile range of the Shannon alpha diversity index of the pregnant woman's vaginal fluid sample is 2.1 or more, the pregnant woman may expected to give birth prematurely

In another aspect, a method for diagnosing preterm birth comprising detecting microorganisms of at least one genus selected from the group consisting of the genus Ureaplasma and the genus Prevotella and microorganisms of the genus Lactobacillus from a vaginal fluid sample of the pregnant woman is provided.

The “vaginal fluid sample”, “Ureaplasma”, “Prevotella”, “Lactobacillus”, “preterm birth”, “prediction”, etc. may be within the aforementioned range.

According to one aspect, the method can detect the relative abundance ratio of microorganisms of at least one genus selected from the group consisting of the genus Ureaplasma and the genus Prevotella and the microorganisms of the genus Lactobacillus, preterm birth can be diagnosed by comparing the detected relative abundance ratio with that of normal pregnant women. Therefore, the method can be usefully utilized for diagnosing preterm birth in pregnant women.

In another aspect, a use of an agent for detecting microorganism of at least one genus selected from the group consisting of the genus Ureaplasma and the genus Prevotella, and microorganism of the genus Lactobacillus, from vaginal fluid samples of pregnant women for the preparation of drugs for diagnosing preterm birth in pregnant women is provided.

The “vaginal fluid sample”, “Ureaplasma”, “Prevotella”, “Lactobacillus”, “preterm birth”, “agent to detect”, etc. may be within the above range.

### Advantageous Effects

According to one aspect, if the relative abundance ratio of microorganisms of one or more genera selected from the group consisting of the genus Ureaplasma and the genus Prevotella in the vaginal fluid sample of the pregnant woman is higher than that of normal pregnant women, and the relative abundance ratio of microorganisms of Lactobacillus genus is lower compared to normal pregnant women, preterm birth can be predicted. Therefore, a composition comprising an agent for detecting microorganism of at least one genus selected from the group consisting of the genus Ureaplasma and the genus Prevotella and microorganism of the genus Lactobacillus in a vaginal fluid sample of a pregnant woman then preterm birth prediction methods using the same can be usefully used to quickly and accurately predict preterm birth in pregnant women.

### DESCRIPTION OF DRAWINGS

FIG. 1 shows a flow chart of experiments for microbiome analysis in vaginal fluid. CVF refers to cervicovaginal fluid, rRNA refers to ribosomal RNA, and OTU refers to an operational taxonomic unit.

FIG. 2a shows a Krona chart depicting microbial taxa in the PTB group at the genus level.

FIG. 2b shows a Krona chart showing microbial taxa in the PTB group at the species level.

FIG. 3a shows a Krona chart depicting microbial taxa in the TB group at the genus level.

FIG. 3b shows a Krona chart depicting the Mycoplasmataceae family among the microbial taxa in the TB group.

FIG. 3c shows a Krona chart showing the Bacteroidetes phylum and Bacteroidales order among the microbial taxa in the TB group.

FIG. 3d shows a Krona chart showing Bacilli classes among microbial taxa in the TB group.

FIG. 3e shows a Krona chart showing Bacillales order among microbial taxa in the TB group.

FIG. 3f shows a Krona chart showing the Proteobacteria phylum among the microbial taxa in the TB group.

FIG. 3g shows a Krona chart depicting microbial taxa in the TB group at the species level.

FIGS. 4a to 4c show the Shannon α-diversity index measured in the PTB group and the TB group.

FIG. 5 shows the sensitivity and specificity of the ROC curve of the Shannon α-diversity index measured in the PTB group and the TB group.

FIG. 6a shows a principal coordinate analysis plot for beta diversity measured in the PTB group and the TB group.

FIG. 6b shows a non-metric multidimensional scaling plot for beta diversity measured in the PTB group and the TB group.

FIG. 7 shows a heat map of microbial taxa of subjects according to CST (community-state type) type.

FIGS. 8a to 8e show the results of comparing the median value of the top 20 microorganisms in the PTB group with the median value of those 20 microoranisms in the TB group.

FIG. 9 shows the differential dominant relative abundance of 13 microorganisms showing significant mean relative abundance ratio between the PTB and TB groups using the Mann-Whitney U test.

FIG. 10 shows the differential dominant relative abundance of 23 microorganisms measured in the PTB group and the TB group by LDA (Linear Discriminant Analysis) effect size analysis.

FIG. 11a is a pattern search result using correlation analysis between microbiome, and shows the top 25 genera having a high correlation with the genus Staphylococcus.

FIG. 11b shows the pattern search results using correlation analysis between microbiome, and shows the top 25 genera with high correlation with the genus Bacteroides.

FIG. 11c is a pattern search result using microbiome correlation analysis, and shows the top 25 genera with high correlation with the genus Sphingomonas.

FIG. 11d is a pattern search result using correlation analysis between microbiome, and shows up to the top 25 genera having a high correlation with the genus Ureaplasma.

FIG. 11e shows the pattern search results using correlation analysis between microbiome, showing the top 25 genera with high correlation with the genus Prevotella.

FIG. 11f is a result of pattern search using correlation analysis between microbiome, and shows the top 25 genera with high correlation with the genus Escherichia.

### MODE FOR INVENTION

Hereinafter, the present invention will be described in more detail through examples. However, these examples are for illustrating the present invention by way of example, and the scope of the present invention is not limited to these examples.

### REFERENCE EXAMPLE

### Reference Example 1. Study Subject and CVF (Cervicovaginal Fluid) Collection

From 2018 to 2020, the subjects were recruited at Ewha Womans University Mokdong Hospital and Yonsei University Severance Hospital receiving approvals from Ewha Womans University Mokdong Hospital Ethics Research Committee (No. 2018-07-007) and Yonsei University Severance Hospital (No. 4-2018-0564). Experiments were performed according to approved guidelines with informed consents from all subjects.

The subjects are women with a single pregnancy and a gestation period between 15 and 36 weeks, asymptomatic pregnant women, and PTL (preterm labor) or hospitalized pregnant women with symptoms of premature rupture of membranes were included, and the subjects with gestational diabetes mellitus, preeclampsia, and lack of medical records were excluded.

CVF samples were collected from the posterior vaginal fornix, using a sterile cotton swab through a speculum exam, prior to vaginal test and clinical treatment with antibiotics, steroids, progesterone and tocolytic. At the time of CVF collection, blood collection was performed for examining the levels of WBC (white blood cell count) and CL (cervical length).

Basic demographic data comprising all pregnant women subjects, age, pre-pregnancy body mass index, education level and preterm birth (PTB) history and health-related characteristics were collected. pregnancy outcomes were assessed by assessing post-delivery, delivery method, GAB (gestational age at birth), newborn body weight, APGAR (appearance, pulse, grimace, activity, respiration) score and neonatal intensive care unit (NICU) hospitalization rates. Those who delivered before 37 weeks were classified into the PTB group, and those who delivered after 37 weeks were classified into the TB group.

### Reference Example 2. Metagenome Analysis Using 16s rRNA Gene Sequencing

To analyze the microbiome of collected CVF samples, NucleoSpin Tissue kit (Macherey-Nagel, Duren, Germany) was used to extract bacterial DNA according to the manufacturer's instructions.

The 16S rRNA sequencing library was constructed according to the 16S metagenome sequencing library preparation protocol targeting the V3 and V4 hypervariable regions of the 16S rRNA gene.

KAPA HiFi HotStart ReadyMix (KAPA Biosystems, Wilmington, USA) and Agencourt AMPure XP system (Beckman Coulter Genomics, Brea, USA) were used for PCR and purification of PCR products, respectively.

The first PCR was performed with 12 ng of template DNA using region-specific primers compatible with Illumina indexes and sequencing adapters. The sequences of these primers are shown in Table 1 below.

TABLE 1

| | |
|---|---|
| Forward | 5′- |
| primer | TCGTCGGCAGCGTCAGATGTGTATAAG |
| | AGACAGTCGTCGGCAGCGTCAGATGT |
| | GTATAAGAGACAGCCTACGGGNGGCW |
| | GCAG-3′ |
| | (Sequence number 1) |
| Reverse | 5′-GTCTCGTGGGCTCGGAGATGTGT |
| primer | ATAAGAGACAGGTCTCGTGGGCTCGG |
| | AGATGTGTATAAGAGACAGGACTACH |
| | VGGGTATCTAATCC-3′ |
| | (Sequence number 2) |

After purification of the PCR products with magnetic beads, a second PCR was performed using primers from the Nextera XT Index kit (Illumina, San Diego, USA) in a limited cycle. Purified PCR products were then visualized using gel electrophoresis and quantified with DropSense96 (Trinean, Gentbrugge, Belgium). Pooled samples were analyzed with an Agilent 2100 Bioanalyzer (Agilent, Santa Clara, Calif., USA) for quality analysis prior to sequencing. Library was quantified by qPCR using the CFX96 real-time system. After normalization, sequencing of the prepared library was performed using the MiSeq system (Illumina, San Diego, USA) with 300 bp paired-end reads. Sequencing data were assembled using QIIME1.9.1 to tag paired-end reads according to overlapping relationships. In the pretreatment step, primers were removed, demultiplexed and filtered for quality (Phred≥20). USEARCH7 was used to perform denoising and chimera detection, filtering in the OTU (Operational Taxonomic Unit) group. Then, OTUs with a similarity of 97% were determined using UCLUST and proximity reference analysis methods through Silva132 and NCBI databases, and OTU identifiers were determined. A comparative OTU task was performed with the database in terms of phylum, class, order, family, genus and species individually using the RDP classifier. Afterwards, α-diversity was analyzed with the Shannon index using QIIME to identify the local population of the microbiome, and by analyzing β-diversity, correlations between other factors and microorganisms were estimated by Bray-Curtis.

Statistical analysis was performed by t-test, Mann-Whitney U test, Chi-square test, Fisher's exact test, heat map, Krona chart, LDA (Linear Discriminant Analysis) effect size (LEfSe), etc., Multivariate analysis, such as non-quantitative multidimensional scaling and principal coordinates analysis was performed. The adjusted P value was calculated by adjusting the false positive rate using the false discovery rate. Correlations between a taxon and a sample groups were analyzed using the Pearson correlation coefficient r as a measure of distance. Statistical analysis was performed using R software (version 3.6.2) and microbiome analysis was performed using MicrobiomeAnalyst (https://www.microbiomeanalyst.ca/) and Calypso (http://cgenome.net/Calypso/) software.

### EXAMPLE

### Example 1. Subject Classification and Clinical Characteristics Comparison

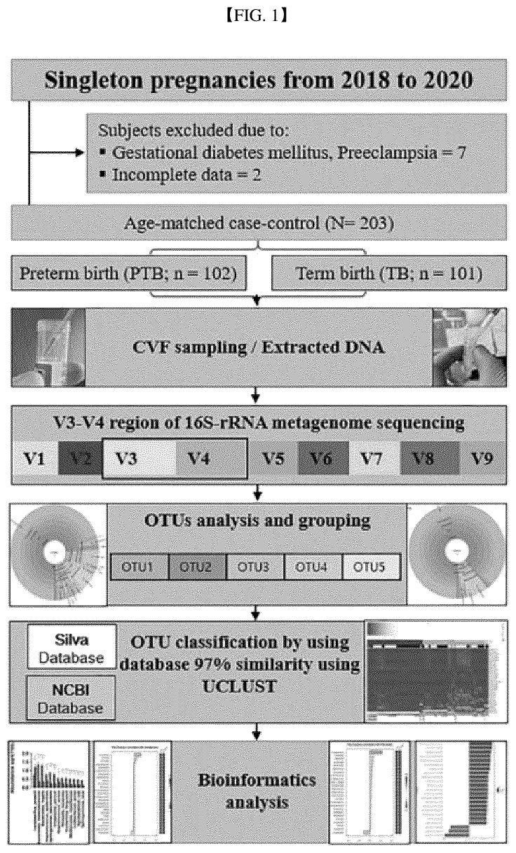

An experiment was conducted with a total of 203 subjects, excluding 7 subjects diagnosed with gestational diabetes mellitus or preeclampsia and 2 subjects with insufficient medical records among the subjects. Of these, 102 were classified into preterm-birth group (PTB; pre-term birth), and 101 were classified into the term birth (TB) group (see FIG. 1).

There was a significant difference (P<0.001) between PTB group and TB group regarding a history of spontaneous PTB (sPTB), White blood cell count (WBC), cervical length (CL), gestational age at sampling (GAS), GAB (gestational age at birth), birth weight, skin color, APGAR (appearance, pulse, grimace, activity, respiration) score and NICU ((neonatal intensive care unit admission) admission rate. However, they did not show any significant difference in characteristics such as maternal age, pre-pregnancy BMI, education level, community status type (CST), normal delivery (ND), and cesarean section (CS). Comparative results of specific clinical characteristics are as follows as shown in Table 2.

TABLE 2

| | Preterm birth | | Term birth | | |
|---|---|---|---|---|---|
| Characteristics | (n = 102) | | (n = 101) | | P-value |
| Maternal age | 32.32 | (±4.35) | 33.02 | (±3.40) | 0.237 |
| (year) | | | | | |
| Pre-pregnancy | 21.60 | (±2.89) | 21.36 | (±2.77) | 0.657 |
| BMI (kg/m²) | | | | | |
| Education level | | | | | >0.999 |
| High school | 6 | (13.3) | 11 | (15.3) | |
| graduation or | | | | | |
| below | | | | | |
| University | 39 | (86.7) | 61 | (84.7) | |
| graduates | | | | | |
| History of PTB | | | | | <0.015* |
| No | 79 | (88.8) | 94 | (97.9) | |
| Yes | 10 | (11.2) | 2 | (2.1) | |
| WBC | 10.1 | (8.8-12.9) | 8.9 | (7.8-10.4) | <0.001* |
| Cervical length | 20.8 | (±12.1) | 30.4 | (±8.5) | <0.001* |
| GAS (weeks) | 31.8 | (26.2-34.2) | 24.5 | (22.1-31.0) | <0.001* |
| CST | | | | | 0.156 |
| I | 46 | (45.1) | 45 | (44.6) | |
| II | 1 | (1.0) | 4 | (4.0) | |
| III | 20 | (19.6) | 22 | (21.8) | |
| IV-A | 7 | (6.9) | 1 | (1.0) | |
| IV-B | 23 | (22.5) | 27 | (26.7) | |
| V | 5 | (4.9) | 2 | (2.0) | |
| Delivery mode | | | | | 0.004* |
| ND | 41 | (40.2) | 61 | (60.4) | |
| CS | 61 | (59.8) | 40 | (39.6) | |
| GAB (weeks) | 34.0 | (30.4-35.6) | 39.3 | (38.2-39.9) | <0.001* |
| Birth weight | 1975.4 | (±780.5) | 3234.9 | (±316.8) | <0.001* |
| (g) | | | | | |
| APGAR score | 6.99 | (5-9) | 9.31 | (9-10) | <0.001* |
| at 1 min | | | | | |
| APGAR score | 8.29 | (7-10) | 9.74 | (10-10) | <0.001* |
| at 5 min | | | | | |
| NICU | 86 | (84.3) | 13 | (12.9) | <0.001* |
| admission | | | | | |

Categorical variables were expressed as frequencies and percentages and analyzed using the chi-square test and Fisher's exact test; continuous variables were expressed as mean±standard deviation (SD) or median (interquartile range) and compared using t-tests or Mann-Whitney U test. CST-I corresponds to Lactobacillus crispatus, CST-II corresponds to Lactobacillus gasseri, CST-III corresponds to Lactobacillus iners, CST-IV-A corresponds to Heterogeneous type, other Lactobacillus spp., and CST-IV-B corresponds to fewer lactobacilli and more anaerobic bacterial taxa, and CST-V corresponds to Lactobacillus jensenii. Statistical significance was defined as P<0.05.

### Example 2. Confirmation of Differences in Microbial Diversity Between the PTB Group and the TB Group

Analysis was performed using the Krona chart, Shannon's α-diversity index, and CST type to identify differences in microbial diversity between the PTB and TB groups.

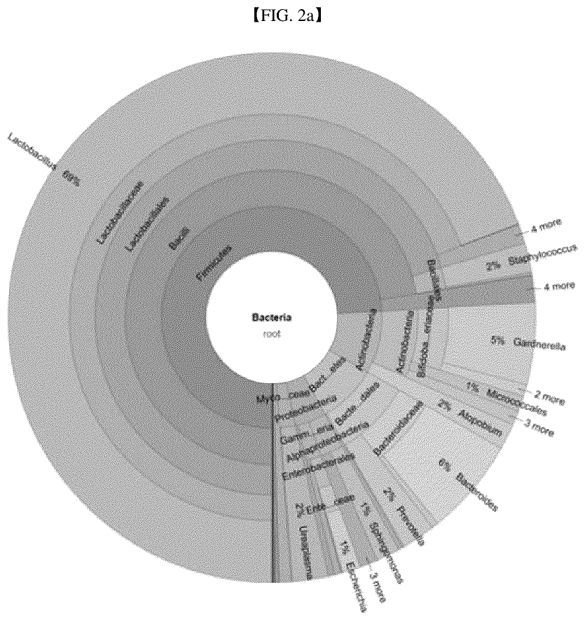

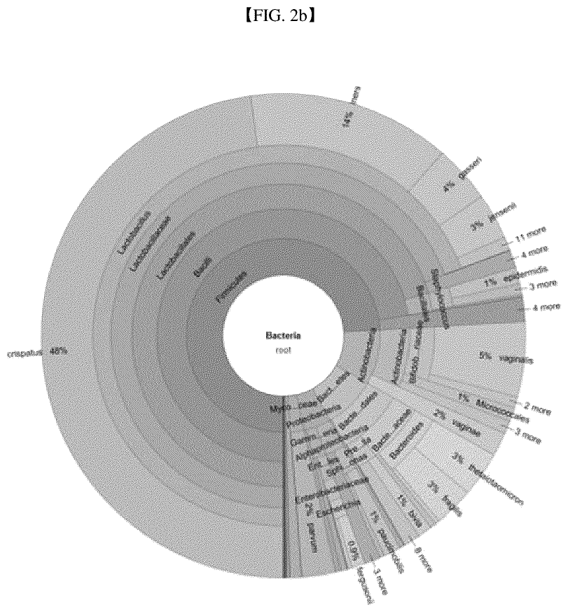

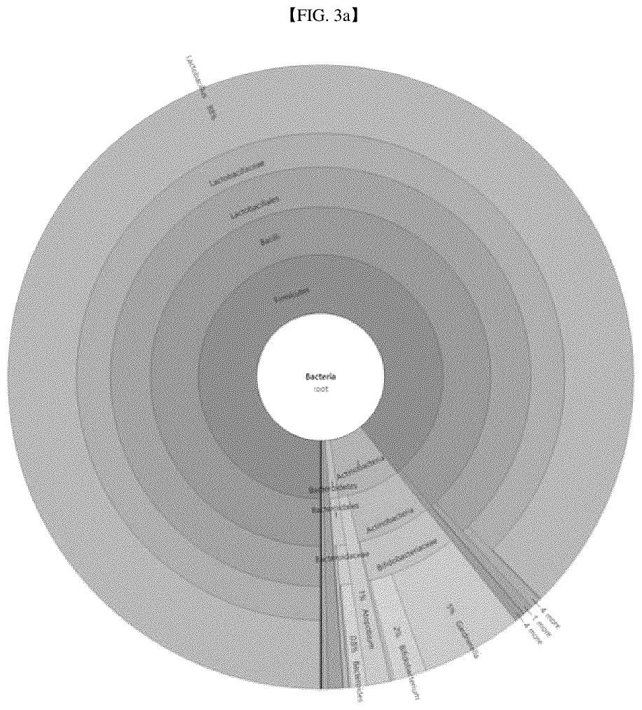

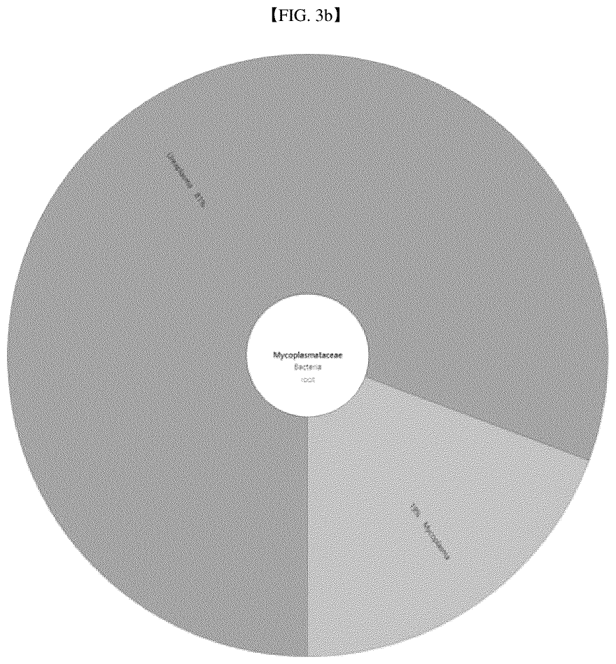

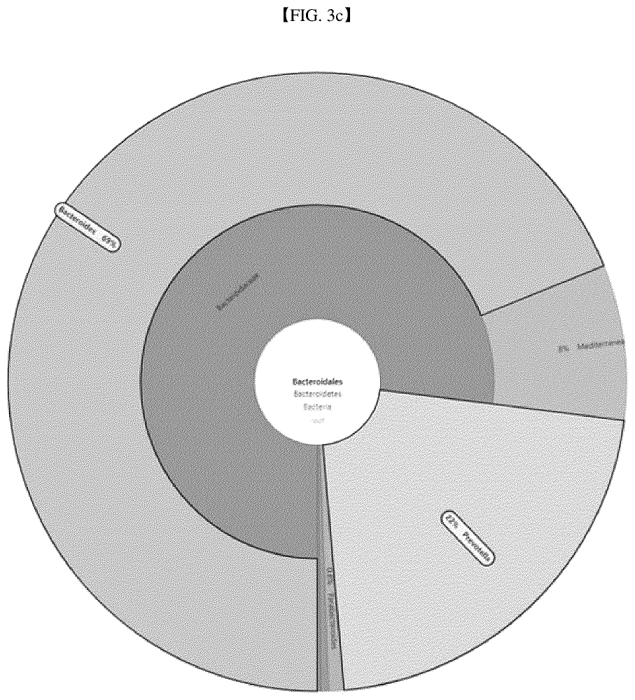

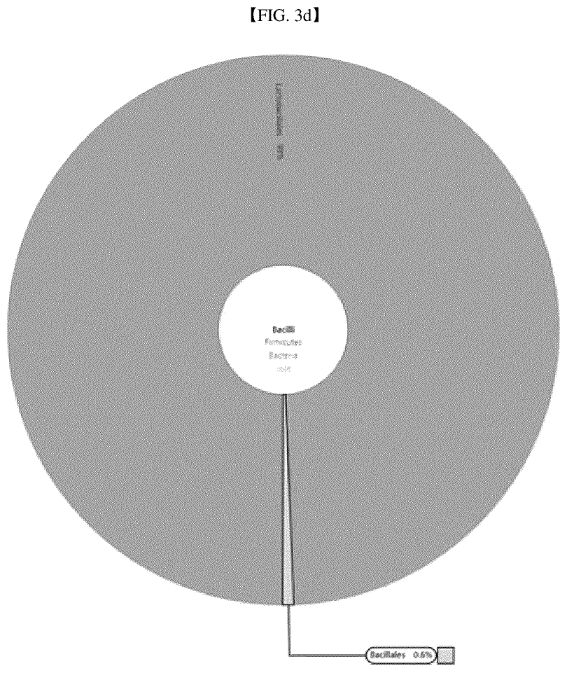

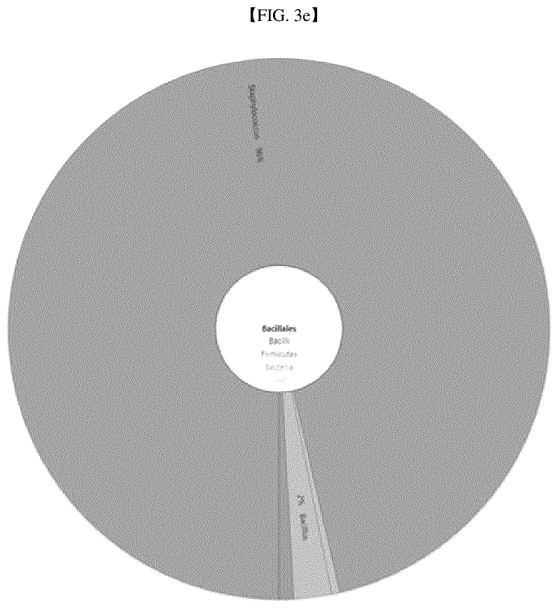

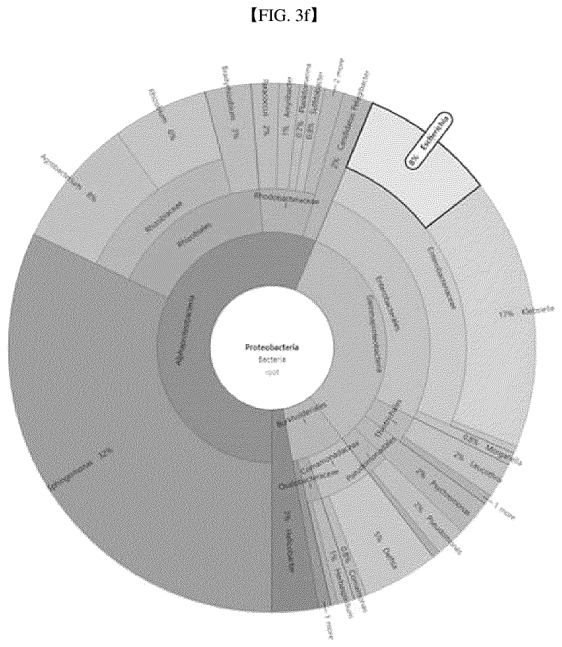

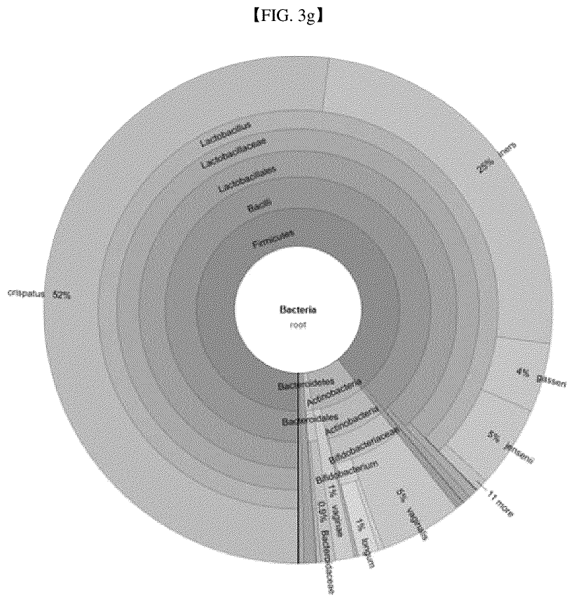

As a result of the Krona chart analysis, it was confirmed that Bacteroidetes, Proteobacteria, and Mollicutes were variously distributed at the phylum level in addition to Firmicutes and Actinobacteria in the PTB group. Specifically, in the PTB group, Lactobacillus accounted for 69%, Prevotella 2%, and Ureaplasma 2% (see FIGS. 2a and 2b), but in the TB group, Lactobacillus accounted for 88%, Prevotella 0.3%, and Ureaplasma. accounted for 0.2%, Staphylococcus accounted for 0.5%, and Escherichia accounted for less than 0.1% (see FIGS. 3a to 3g).

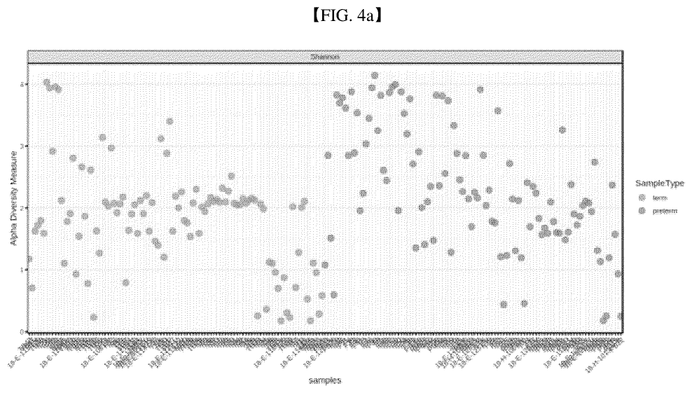

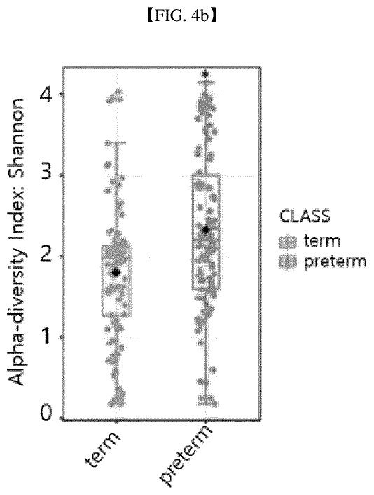

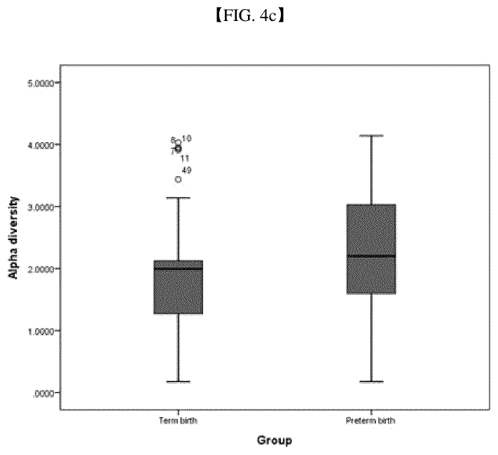

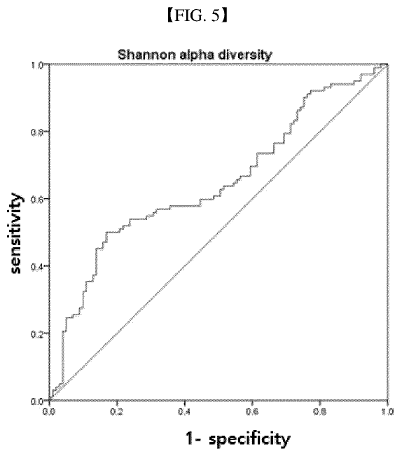

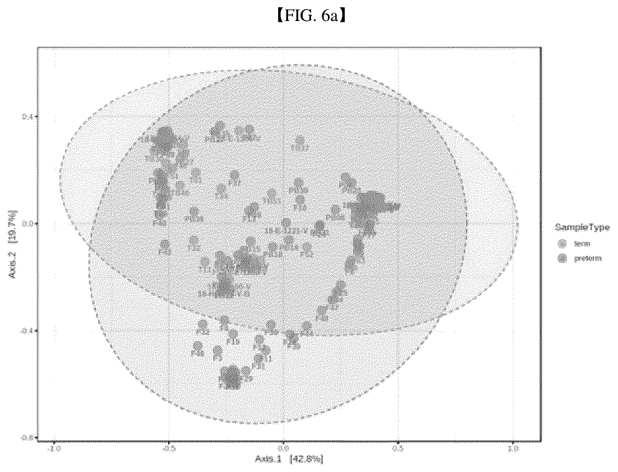

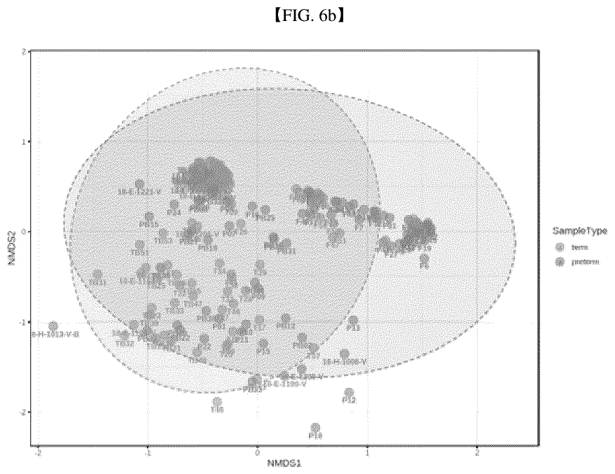

As a result of Shannon's α-diversity index analysis, the median value (interquartile range) of the PTB group was 2.20 (1.27-2.12), which was significantly higher than the median value (interquartile range) of the TB group, 1.99 (1.60-3.03). (See FIGS. 4a to 4c). As a result of drawing and analyzing the ROC curve with Shannon's α-diversity index, the AUC value was 0.65, with a sensitivity of 50% and specificity of 83%, indicating it was confirmed that there was a significant difference in the α-diversity index between the PTB and TB groups (see Table 3 and FIG. 5). In the case of β-diversity analysis, most of the microbial communities were similar, but it was confirmed that there were differences in the degree of similarity between specific microbes (see FIGS. 6a and 6b).

TABLE 3

| Cut-off | Sensitivity | | Specificity | | AUC |
|---|---|---|---|---|---|
| 2.20954 | 0.5 | 0.168 | 0.832 | 0.332 | 0.646 |

On the other hand, as a result of analyzing the CST types using a heat map, no significant difference between the PTB group and the TB group was confirmed (see FIG. 7).

Through the above experiments, it was confirmed that there was a significant difference in the α-diversity index between the PTB group and the TB group. It was confirmed that the PTB group had a lower ratio of Lactobacillus than that of the TB group while the ratio of Prevotella and Ureaplasma was higher than that of the TB group.

### Example 3. PTB Group and TB Group Related Microbiome Analysis

The related microbiome in each of the PTB and TB groups was analyzed using a modified P value, average relative abundance expressed as total sum scaling (TSS), and LDA score through linear discriminant analysis (LDA).

As a result of calculating statistical significance by Mann-Whitney U test using the corrected P value by adjusting the false positive rate through the false discovery rate, a total of 157 bacterial species in the two groups were detected, of these, 82 bacteria had a corrected P value of less than 0.05.

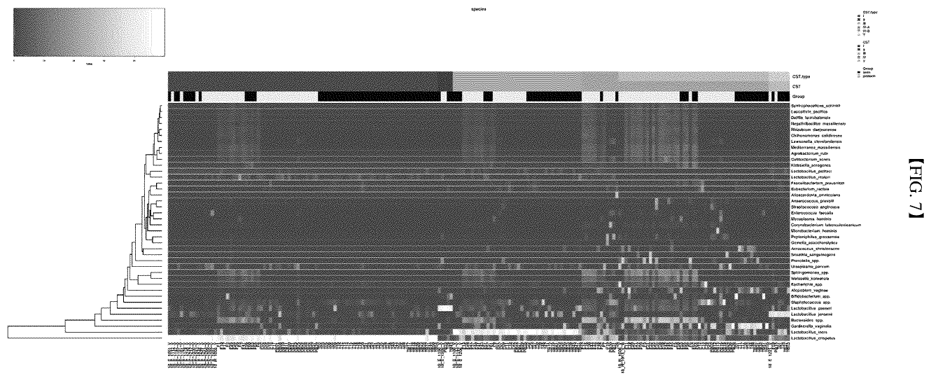

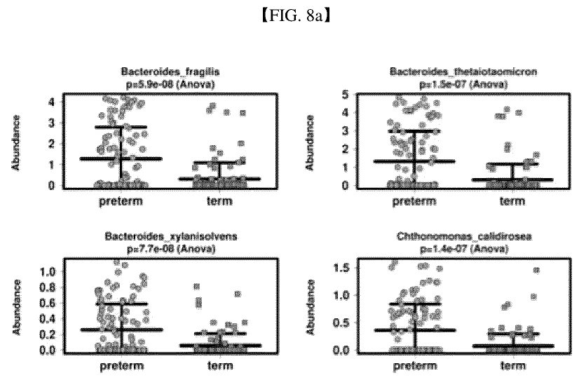

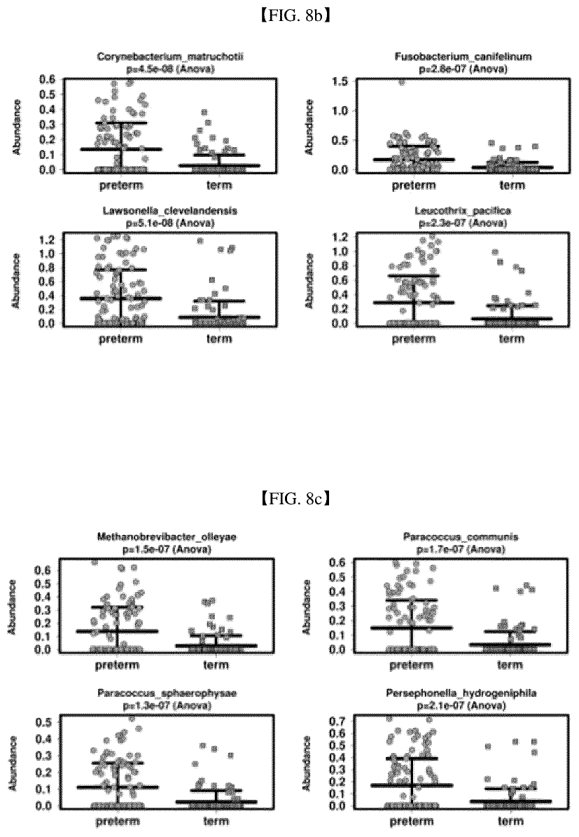

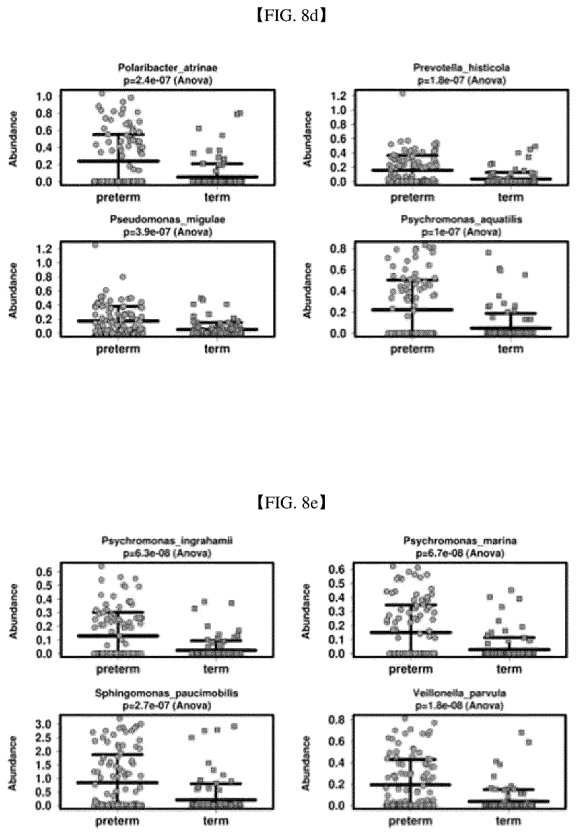

Among them, bacteria with a median value of the top 20 in the PTB group were Bacteroides fragilis, Bacteroides thetaiotaomicron, Bacteroides xylanisolvens, Chthonomonas calidirosea, Corynebacterium matruchotii, Fusobacterium canifelinum, Lawsonella clevelandensis, Leucothrix pacifica, Methanobrevibacter olleyae, Paracoccus communis, Paracoccus sphaerophysae, Persephonella hydrogeniphila, Polaribacter atrinae, Prevotella histicola, Pseudomonas migulae, Psychromonas aquatilis, Psychromonas ingrahamii, Psychromonas marina, Sphingomonas paucimobilis and Veillonella parvula, and the median value in the PTB group of the bacteria was compared with the median value in the TB group (see FIGS. 8a to 8e).

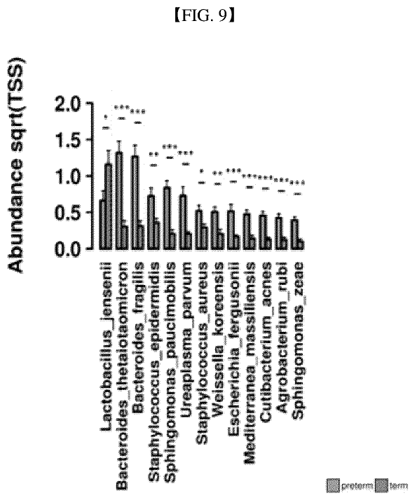

In addition, as a result of analyzing the significant mean relative abundance determined using the Mann-Whitney U test by total sum scaling (TSS), it is confirmed that a total of 13 bacteria were significantly different between the PTB and TB groups (abundance>0.5%). Specifically, the abundance of Lactobacillus jensenii was significantly higher in the TB group than in the PTB group, and in the PTB group, the abundance of Bacteroides thetaiotaomicron, Bacteroides fragilis, Staphylococcus epidermidis, Sphingomonas paucimobilis, Ureaplasma parvum, Staphylococcus aureus, Weissella koreensis, Escherichia fergusonii, Mediterranea massiliensis, Cutibacterium acnes, Agrobacterium rubi, and Sphingomonas zeae were significantly higher than those in the TB group. (see FIG. 9).

Additionally, using LDA (linear discriminant analysis), differential dominant relative abundance of bacterial subtaxa was measured. and the result was that total of 23 species appeared with a significant difference (P<0.05, LDA score≥4).

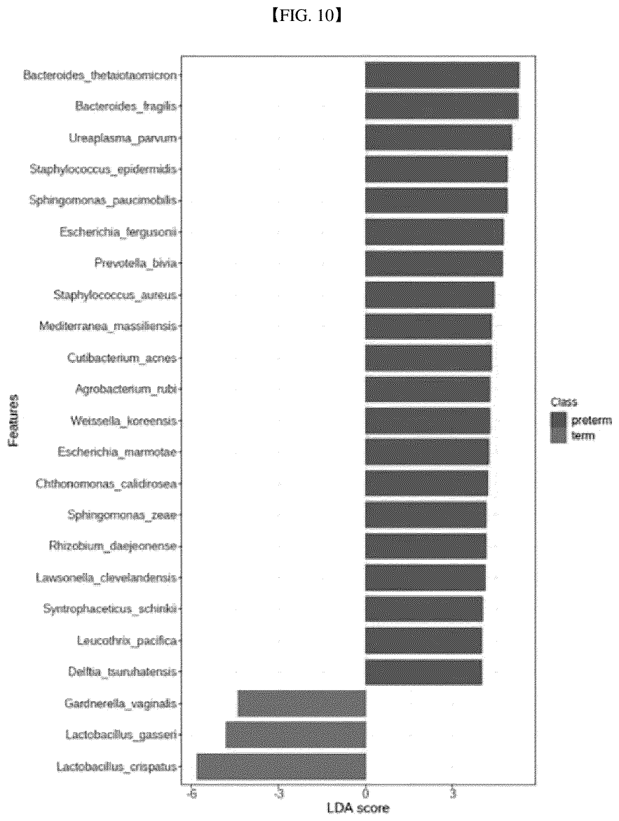

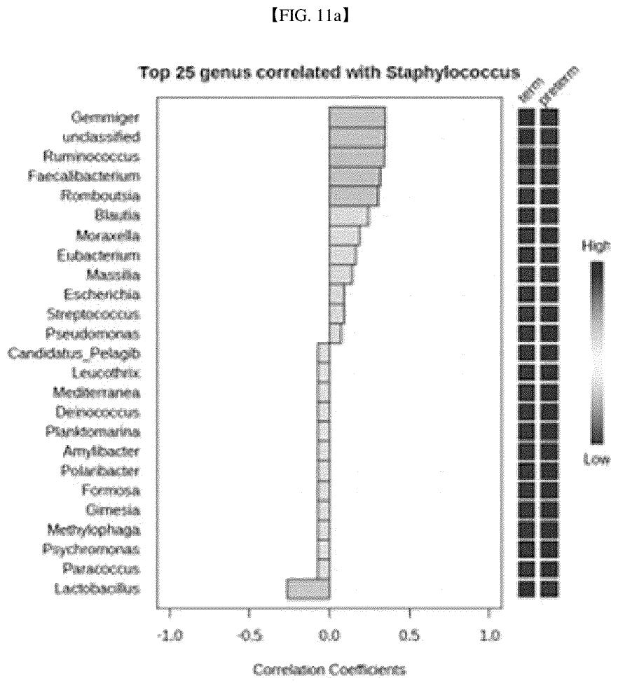

Specifically, in the TB group, 3 species of bacteria such as Lactobacillus crispatus, Lactobacillus gasseri and Gardnerella vaginalis are the main taxa, and in the PTB group, 20 species bacteria such as Bacteroides thetaiotaomicrom, Bacteroides fragilis, Ureaplasma parvum, Staphylococcus epidermidis, Sphingomonas paucimobilis, Escherichia fergusonii, Prevotella bivia, Staphylococcus aureus, Mediterranea massiliensis, Cutibacterium acnes, Agrobacterium rubi, Weissella koreensis, Escherichia marmotae, Chthhonomonas calidirosea, Sphingomonas zeae, Rhizobium daejeonense, Lawsonella clevelandensis, Syntrophaceticus schinkii, Leucothrix pacifica and Delftia tsuruhatensis were appeared as major taxa (see FIG. 10).

Through the above experiments, bacterial species mainly present in the PTB group and the TB group were specifically identified.

### Example 4. Correlation Analysis Between Microbiome in PTB Group and TB Group

Analysis was performed at the representative genus level using correlation analysis to identify significant differences in microbiome patterns.

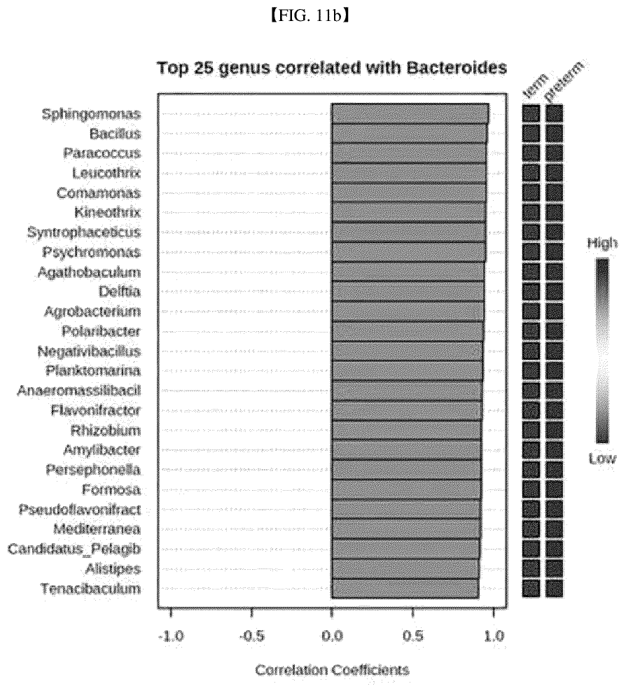

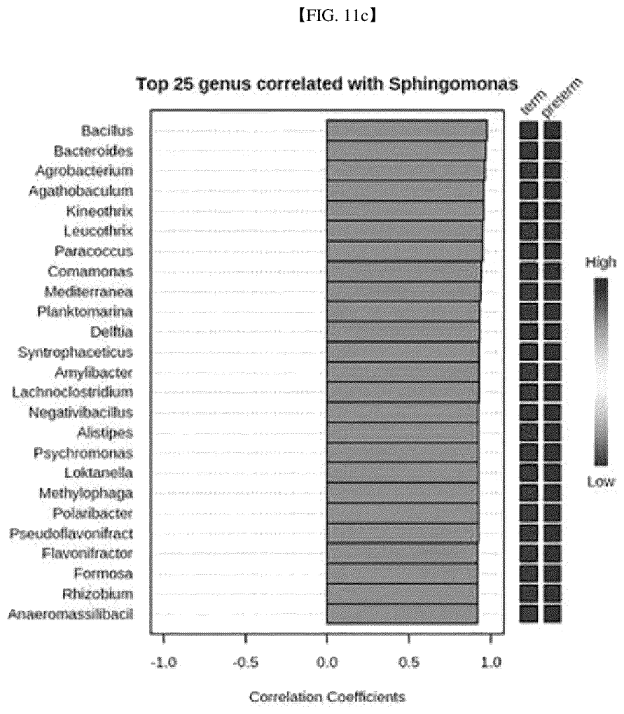

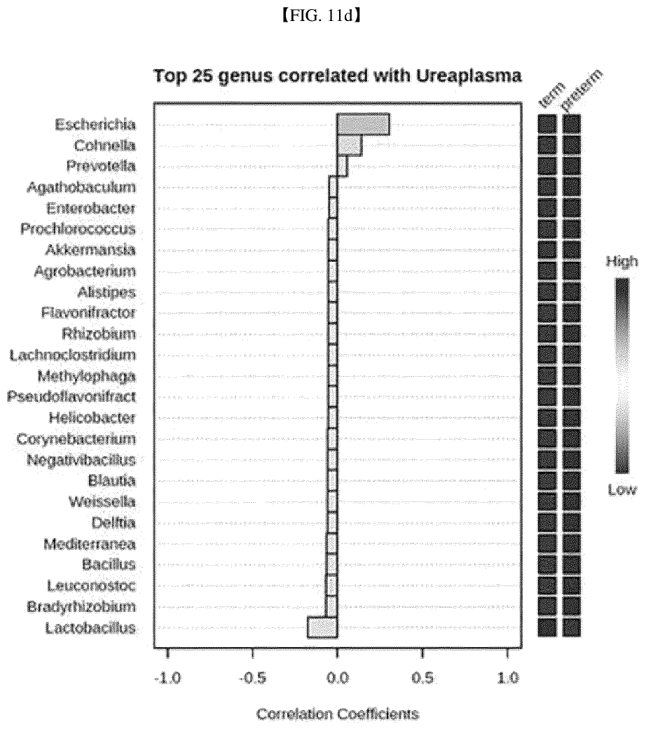

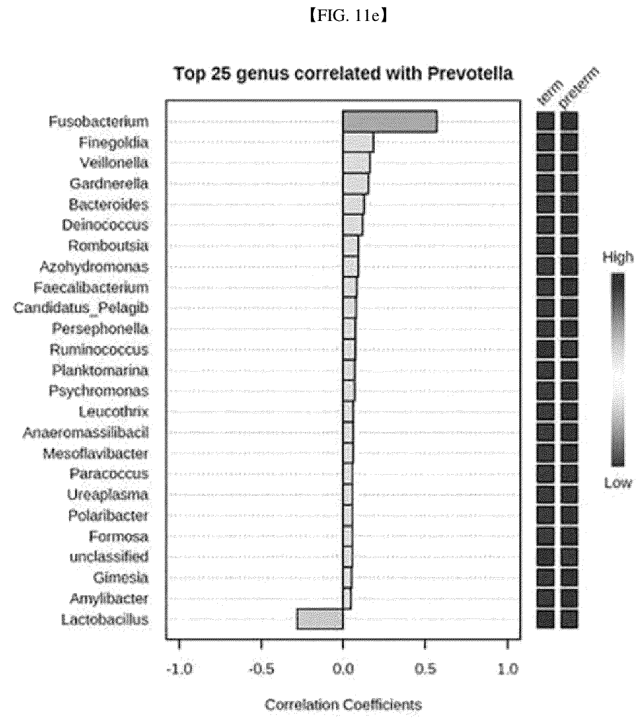

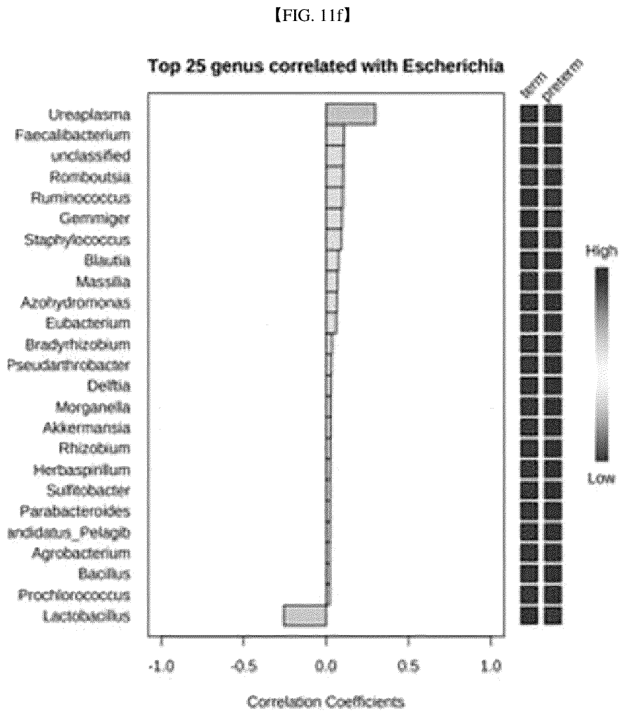

As a result of the analysis, Staphylococcus showed a positive correlation of 0.3 with Gemmiger and Ruminococcus, and a negative correlation of 0.3 with Lactobacillus, indicating an association with TB in this case (see FIG. 11a).

Bacteroides showed a positive correlation of 0.8 or more with 43 genera and a negative correlation of 0.6 with Lactobacillus (see FIG. 11b), and Sphingomonas showed a positive correlation of 0.8 or more with 46 genera and showed a negative correlation of 0.6 with Lactobacillus. (see FIG. 11c).

Ureaplasma showed a correlation of 0.3 with Escherichia, and Prevotella showed a positive correlation of 0.5 with Fusobacterium (see FIGS. 11d and 11e). When Ureaplasma showed a negative correlation with Lactobacillus, it showed a high correlation with TB. Similarly, in the case of Prevotella, a negative correlation with Lactobacillus showed a high correlation with TB. In addition, the positive correlation between Prevotella and Ruminococcus also showed a high correlation with TB.

Escherichia showed a positive correlation with Gemmiger and Ruminococcus, and a negative correlation with Lactobacillus, and in this case, the correlation with TB was high (see FIG. 11f).

Through the above vaginal microbiome correlation analysis experiment, it was verified that the correlation between microbial communities, not just the presence of specific bacteria in the vagina, can determine preterm birth and full term birth. That is, Ureaplasma and Prevotella have been known as pathogenic microorganisms that cause preterm birth, but by confirming that they do not cause complications such as preterm birth when the relative abundance ratio of those microorganisms and Lactobacillus is equal to or lower than the normal case, it was confirmed that the balance of Lactobacillus, Ureaplasma, and Prevotella played an important role in preventing preterm birth.

1. A composition for predicting preterm birth of the pregnant woman comprising an agent for detecting microorganisms of at least one genus selected from the group consisting of the genus Ureaplasma and the genus Prevotella and microorganisms of the genus Lactobacillus in a vaginal fluid sample of the pregnant woman.

2. The composition according to claim 1, wherein the agent further detects microorganisms of one or more genera selected from the group consisting of the genus Staphylococcus and the genus Escherichia.

3. The composition according to claim 2, wherein the agent detects the relative abundance ratio of microorganisms of one or more genera selected from the group consisting of the genus Ureaplasma and the genus Prevotella, microorganisms of one or more genera selected from the group consisting of the genus Staphylococcus and the genus Escherichia and microorganisms of the genus Lactobacillus.

4. The composition according to claim 1, wherein the agent is at least one selected from the group consisting of primers, probes, antisense oligonucleotides, aptamers and antibodies specific to the microorganisms.

5. The composition according to claim 1, wherein the agent is specific to one or more base sequences selected from the group consisting of 16s rRNA base sequence, 18s rRNA base sequence and ITS (Internal transcribed spacer) base sequence of the microorganisms.

6. The composition according to claim 1, wherein the agent further confirms the Shannon α-diversity index.

7. A kit for predicting preterm birth of the pregnant woman comprising an agent for detecting microorganisms of at least one genus selected from the group consisting of the genus Ureaplasma and the genus Prevotella and microorganisms of the genus Lactobacillus in a vaginal fluid sample of the pregnant woman.

8. The kit according to claim 7, wherein the agent further detects microorganisms of at least one genus selected from the group consisting of the genus Staphylococcus and the genus Escherichia.

9. A method providing information for predicting preterm birth of the pregnant woman, comprising the step of detecting microorganisms of at least one genus selected from the group consisting of the genus Ureaplasma and the genus Prevotella, and microorganisms of the genus Lactobacillus, from a vaginal fluid sample isolated from the pregnant woman.

10. The method according to claim 9, wherein when the relative abundance ratio of the detected microorganisms of at least one genus selected from the group consisting of the genus Ureaplasma and the genus Prevotella is lower than that of normal pregnant woman, and
the relative abundance ratio of the detected microorganisms of the genus Lactobacillus is higher than that of normal pregnant woman, it is predicted that full-term birth will occur,
when the relative abundance ratio of the detected microorganisms of at least one genus selected from the group consisting of the genus Ureaplasma and the genus Prevotella is higher than that of normal pregnant woman, and
the relative abundance ratio of the detected microorganisms of the genus Lactobacillus is lower than that of normal pregnant woman, it is predicted that preterm birth will occur.

11. The method according to claim 9, wherein the method further comprises step of detecting microorganisms of at least one genus selected from the group consisting of the genus Staphylococcus and the genus Escherichia.

12. The method according to claim 11, wherein when the relative abundance ratio of the detected microorganisms of at least one genus selected from the group consisting of the genus Ureaplasma and the genus Prevotella is lower than that of normal pregnant woman,
the relative abundance ratio of the detected microorganisms of at least one genus selected from the group consisting of the genus Staphylococcus and the genus Escherichia is lower than that of normal pregnant woman, and
the relative abundance ratio of the detected microorganisms of the genus Lactobacillus is higher than that of normal pregnant woman, it is predicted that full-term birth will occur,
when the relative abundance ratio of the detected microorganisms of at least one genus selected from the group consisting of the genus Ureaplasma and the genus Prevotella is higher than that of normal pregnant woman,
the relative abundance ratio of the detected microorganisms of at least one genus selected from the group consisting of the genus Staphylococcus and the genus Escherichia is higher than that of normal pregnant woman, and
and the relative abundance ratio of the detected microorganisms of the genus Lactobacillus is lower than that of normal pregnant woman, it is predicted that preterm birth will occur.

13. The method according to claim 9, wherein the method further comprises step of confirming the Shannon α-diversity index from the sample.

14. The method according to claim 13, wherein when the relative abundance ratio of the detected microorganisms of at least one genus selected from the group consisting of the genus Ureaplasma and the genus Prevotella is lower than that of normal pregnant woman,
the relative abundance ratio of the detected microorganisms of the genus Lactobacillus is higher than that of normal pregnant woman, and
the Shannon α-diversity index identified above is equal to or smaller than the Shannon α-diversity index of normal pregnant woman, it is predicted that full-term birth will occur,
when the relative abundance ratio of the detected microorganisms of at least one genus selected from the group consisting of the genus Ureaplasma and the genus Prevotella is higher than that of normal pregnant woman,
the relative abundance ratio of the detected microorganisms of the genus Lactobacillus is lower than that of normal pregnant woman, and
the Shannon α-diversity index identified above is larger than the Shannon α-diversity index of normal pregnant woman, it is predicted that preterm birth will occur.


SEQUENCE LISTING (2 sequences)

SEQ ID NO: 1 - DNA, 83 bp, synthetic construct
  misc_feature 1..83: note = primer
  misc_difference 75replace
  source 1..83: mol_type = other DNA
tcgtcggcagcgtcagatgtgtataagagacagtcgtcggcagcgtcagatgtgtataagagacagcctacgggnggcwgcag

SEQ ID NO: 2 - DNA, 89 bp, synthetic construct
  misc_feature 1..89: note = primer
  source 1..89: mol_type = other DNA
gtctcgtgggctcggagatgtgtataagagacaggtctcgtgggctcggagatgtgtataagagacaggactachvgggtatctaatcc